(19) United States
(12) Patent Application Publication
(10) Pub. No.: US 2019/0029933 A1
(43) Pub. Date: Jan. 31, 2019

(54) Activated Carbon Relaxer System

(71) Applicant: Rafe Williams, JR., Atlanta, GA (US)

(72) Inventor: Rafe Williams, JR., Atlanta, GA (US)

(21) Appl. No.: 16/024,894

(22) Filed: Jul. 1, 2018

Related U.S. Application Data
(60) Provisional application No. 62/537,459, filed on Jul. 27, 2017.

(51) Int. Cl. A61K 8/19; B01J 20/20; A61Q 5/04; A61K 8/43

(52) U.S. Cl. CPC A61K 8/19; B01J 20/20; A61K 2800/75; A61K 8/43; A61Q 5/04

(57) ABSTRACT

Straightening the hair with the activated carbon relaxer system drastically reduces or, eliminates post perm hair odor (PPO) caused by lanthionization, which occurs in all hydroxide relaxer systems. During the relaxing process, the activated carbon component of the compound directly absorbs the malodorous sulfur gases and byproducts that are released from deep within the hair fiber. After allowing the relaxer system adequate processing time to straighten the hair, the compound along with the absorb sulfur gases and byproducts, are removed from the hair by rinsing the hair with water and neutralizing shampoo, thereby preventing the released gases from entering the air and causing PPO.

### CROSS-REFERENCE TO RELATED APPLICATIONS

62/537,459 Jul. 27, 2017 Rafe Williams JR.

### BACKGROUND OF THE INVENTION

### Field of the Invention

This invention relates to the field of chemical hair straightening and, in particular hydroxide relaxer formulations. Activated carbon is added to hydroxide relaxer formulations to reduce or eliminate the so-called “post-perm-odor” which occurs as a result of lanthionization.

### Description

Presently in hydroxide relaxer systems, when the disulfide bonds are broken by the acting hydroxide, sulfur gas is released that result in a malodorous chemical smell that remains in the hair and is known as post-perm-odor (PPO). Although most clients accept the unpleasant PPO as an unavoidable part of the perming process, there are many potential clients who choose not to get their hair permed (relaxed) because of it.

Over the years there have been many remedies that claim to reduce the so called PPO, such as adding fragrances to the relaxer formulation in an attempt to mask the smell or requiring the technician to perform additional shampooing and rinsing after the relaxer process has been completed or, in some cases, the technicians advise their clients to rinse their hair with tomato juice, or vinegar three or four days after their relaxer service. The belief is that the lower pH values of these household items will further neutralize the permed hair and therefore further reduce the PPO. Although a few of these remedies slightly work, they, by no means, drastically reduce or eliminate PPO and in most cases inconvenience their clients.

### BRIEF SUMMARY OF THE INVENTION

All hydroxide relaxer systems are ionic compounds formed by the acting hydroxides OH of metals such as sodium hydroxide (NaOH), potassium hydroxide (KOH), lithium hydroxide (LiOH), calcium hydroxide (CaOH) and guanidine hydroxide (CH6N3O). Although the pH, color, viscosity and texture (emulsions, lotions, creams, gels, paste or liquids), may vary from one formulation to the next, depending on the design goals of the formulator, the actual relaxing process (lanthionization) remains the same and is caused by the highly alkaline hydroxide compound breaking the disulfide bonds in the hair fiber thereby leaving the hair relaxed (straight) with an unpleasant PPO.

It has now been found that activated carbon, also called activated charcoal, which is a form of charcoal processed to have small low-volume pores that increase the surface area available for absorption, when added to any hydroxide relaxer formulation, the amount of PPO can be dramatically reduce or eliminated as determined by the percent of activated carbon addition to the total batch weight with negligible effects on the processing and, in some formulation it actually enhances the performance. Unlike the addition of fragrances to the relaxer system, where the PPO is being masked with a pleasant smell, the activated carbon, which is undisputedly known for its ability to absorb odorous gases such as sulfur, actually absorbs the malodorous gas. Since the activated carbon is part of the processing compound it absorbs the odorous gases before they are released into the surrounding air thereby reducing or eliminating PPO at the source.

### DETAILED DESCRIPTION AND IMPLEMENTATION

The odors occurring during and after a hydroxide relaxer service primarily result from the sulfur gases released in the air by lanthionization. Lanthionization is the process by which hydroxide relaxers permanently straightens hair. The hydroxide ions in the compound remove sulfur atoms from the disulfide bond in the hair fibers and convert them into a lanthionine bonds. Since a disulfide bond consist of two bonded sulfur atoms, and the lanthionine bond only contains one sulfur atom, the second sulfur atom is released into the air and combines with oxygen and water vapor to form sulfur oxides SO₂ and hydrogen sulfide H₂S which causes the unpleasant smell in the client's hair. When activated carbon is added to a hydroxide compound, and the hair fibers are properly covered by the compound during the relaxing process, any sulfur gases released during the process are immediately absorbed by the activated carbon in the relaxer compound and are not available to form the malodorous gases (SO₂ or H₂S). Once the straightening process is complete, the activated carbon compound is neutralized and rinsed from the hair in the same manner as legacy hydroxide relaxer systems. However, in the case of the activated carbon compound, the malodorous gases that were absorbed are rinsed and shampooed from the hair along with the processed compound, leaving the hair free of PPO.

Although there are many benefits to adding activated carbon to hydroxide relaxer systems there is one characteristic that must be noted. To reduce or eliminate PPO in hydroxide relaxer systems, activated carbon must be present in the compound in amounts greater than 0.1% of the total batch weight. Since activated carbon is naturally black in color, when 1.0% or more of activate carbon is added to a hydroxide relaxer batch it drastically changes the compound's color from and white or egg-shell to grey or black as determined, by the amount of activated carbon added. The unique dark color of the activated carbon relaxer system is considered to be the primary identifier of its absorption and odorless functionality.

Hydroxide relaxer formulations can be divided into six specific categories: single metal mix-base, single metal mix-no-base, single metal no-mix-base, single metal no-mix-no-base, multi-metal no-mix-base, multi-metal no-mix-no-base. As will be demonstrated in the following examples, with the proper adjustments of the formulation ingredients, activated carbon can be added to any hydroxide category to eliminate PPO.

As stated previously, although the hydroxide relaxer formulations may differ in their ingredients and percentages, they all relax the hair the same. In other words, some formulas are designed to condition the hair during the process while others are designed to be less damaging or irritating to the hair and scalp. Regardless of the formulator's design objective, they remain hydroxide relaxers and utilize lanthionization to straighten the hair.

The following example formulations illustrate the implementation of a few of the hydroxide relaxer categories covered by this invention. Generally, the preferred ingredients are use but are not intended to be limited thereby.

### Example 1: Regular Strength Single Metal No-Mix Base

| INGREDIENTS | | |
|---|---|---|
| INCI | PART | % W/W |
| Petrolatum | A | 19.00 |
| Polybase CT (cetearyl | A | 9.00 |
| alchol, polysorbate 60) | | |
| Ceteareth-20 | A | 0.75 |
| Mineral Oil 70 | A | 9.00 |
| PEG-75 Lanolin 50% | A | 3.00 |
| Activated Charcoal | A | 2.50 |
| Dimethicone 200 | A | 0.12 |
| Plantasil | A | 1.30 |
| Propylene Glycol | B | 3.00 |
| Deionized Water | B | 45.23 |
| Potassium Hydroxide | C | 6.60 |
| 45.5% Solution | | |
| Black Seed Oil | D | 0.50 |
| Total | | 100.00 |

### Example 2: Regular Strength Single Metal No-Mix No-Base

| INGREDIENTS | | |
|---|---|---|
| INCI | PART | % W/W |
| Petrolatum | A | 19.00 |
| Polybase CT (cetearyl | A | 8.15 |
| alchol, polysorbate 60) | | |
| Ceteareth-20 | A | 0.75 |
| Mineral Oil 70 | A | 9.00 |
| PEG-75 Lanolin 50% | A | 3.00 |
| Activated Charcoal | A | 2.50 |
| Glycerin | B | 6.00 |
| Deionized Water | B | 45.00 |
| Potassium Hydroxide | C | 6.60 |
| 45.5% Solution | | |
| Total | | 100.00 |

### Example 3A, Regular Strength Single Metal Mix No-Lye Base

| INGREDIENTS | | |
|---|---|---|
| INCI | PART | % W/W |
| Petrolatum | A | 19.00 |
| Polybase CT (cetearyl | A | 10.40 |
| alchol, polysorbate 60) | | |
| Ceteareth-20 | A | 0.75 |
| Mineral Oil 70 | A | 9.30 |
| PEG-75 Lanolin 50% | A | 3.00 |
| Activated Charcoal | A | 2.50 |
| Dimethicone 200 | A | 0.12 |
| Propylene Glycol | B | 3.00 |
| Deionized Water | B | 46.53 |
| Calcium Hydroxide | B | 5.00 |
| Vitamin E | C | 0.30 |
| Olive Oil | C | 0.10 |
| Total | | 100.00 |

### Example 3B, Regular Strength Single Metal Mix No-Lye No-Base

| INGREDIENTS | | |
|---|---|---|
| INCI | PART | % W/W |
| Petrolatum | A | 19.00 |
| Polybase CT (cetearyl | A | 10.40 |
| alchol, polysorbate 60) | | |
| Ceteareth-20 | A | 0.75 |
| Mineral Oil 70 | A | 9.30 |
| PEG-75 Lanolin 50% | A | 3.00 |
| Activated Charcoal | A | 2.50 |
| Dimethicone 200 | A | 0.12 |
| Glycerin | B | 3.00 |
| Deionized Water | B | 46.53 |
| Calcium Hydroxide | B | 5.00 |
| Vitamin E | C | 0.30 |
| Olive Oil | C | 0.10 |
| Total | | 100.00 |

### Example 3C, Activator for No-Lye Base and No-Base

| INGREDIENTS | | |
|---|---|---|
| INCI | PART | % W/W |
| Dionized Water | A | 72.96 |
| Gylcerine | B | 25.00 |
| Guanidine Carbonate | B | 2.00 |
| EDTA | B | 0.02 |
| FD&C yellow 5 (2%) | B | 0.02 |
| Total | | 100.00 |

### Example 4, Regular Strength Multi-Metal No-Mix Base

| INGREDIENTS | | |
|---|---|---|
| INCI | PART | % W/W |
| Petrolatum | A | 21.00 |
| Polybase CT (cetearyl | A | 9.35 |
| alchol, polysorbate 60) | | |
| Ceteareth-20 | A | 0.75 |
| Mineral Oil 70 | A | 9.00 |
| PEG-75 Lanolin 50% | A | 3.00 |
| Activated Charcoal | A | 2.50 |
| Dimethicone 200 | A | 0.12 |
| Plantasil | A | 1.30 |
| Propylene Glycol | B | 3.00 |
| Deionized Water | B | 45.23 |
| Sodium Hydroxide | C | 1.00 |
| 50% Solution | | |
| Potassium Hydroxide | D | 2.30 |
| 45.5% Solution | | |
| Lithium Hydroxide | E | 1.45 |
| powder | | |
| Total | | 100.00 |

### Example 5, Regular Strength Multi-Metal No-Mix No-Base

| INGREDIENTS | | |
|---|---|---|
| INCI | PART | % W/W |
| Petrolatum | A | 21.00 |
| Polybase CT (cetearyl | A | 9.35 |
| alchol, polysorbate 60) | | |
| Ceteareth-20 | A | 0.75 |
| Mineral Oil 70 | A | 9.00 |
| PEG-75 Lanolin 50% | A | 3.00 |
| Activated Charcoal | A | 2.50 |
| Dimethicone 200 | A | 0.12 |
| Plantasil | A | 1.30 |
| Glycerin | B | 3.00 |
| Deionized Water | B | 45.23 |
| Sodium Hydroxide | C | 1.00 |
| 50% Solution | | |
| Potassium Hydroxide | D | 2.30 |
| 45.5% Solution | | |
| Lithium Hydroxide | E | 1.45 |
| powder | | |
| Total | | 100.00 |

Batching procedure for examples 1, 2, 3A, 3B, 4 and 5: weigh part A into sweep type jacketed tank and begin heating to 78 C. Weigh part B in a separate jacketed tank and begin agitation and heating to 78 C. When both tanks are at temperature, slowly add part B to part A, avoiding aeration. Cool to 60 C and add parts C, D and E. Avoid aeration. Cool to 25 C and pump through colloid mill or versator and package.

Batching procedure for example 3C: weigh part A into jacketed tank and begin agitation and heating to 50 C. Weigh part B ingredients and add to jacketed tank in descending order while continuing agitation. After 20 minutes of agitation, allow cooling to 25 C and package. In this example the BASE and ACTIVATOR must be premixed together at a ratio 5:1.6, to perform as a regular strength carbon relaxer.

Each of the preceding formulations has a specific design goal and was primarily chosen for its batching simplicity.

What is claimed is:

1. A method of adding activated carbon in the amount greater than 0.1% of the total batch weight, into any hydroxide relaxer formulation categories to drastically reduce or eliminate PPO that results from lanthionization.

2. The method of claim 1 for single metal no-mix hydroxide relaxer formulation, base and no base.

3. The method of claim 1 for single metal mix hydroxide relaxer formulation, base and no base.

4. The method of claim 1 for multi-metal no-mix hydroxide relaxer formulation, base and no base.

5. The method of claim 1 for multi-metal mix hydroxide relaxer formulation, base and no base.
